(19) United States
(12) Patent Application Publication
(10) Pub. No.: US 2019/0343205 A1
(43) Pub. Date: Nov. 14, 2019

(54) Self-Adjustment Belt with Bumps

(71) Applicant: Alina Clausen, Tampa, FL (US)

(72) Inventor: Alina Clausen, Tampa, FL (US)

(21) Appl. No.: 16/410,833

(22) Filed: May 13, 2019

Related U.S. Application Data
(60) Provisional application No. 62/670,761, filed on May 12, 2018.

(51) Int. Cl. A41F 9/00; A61H 11/00

(52) U.S. Cl. CPC A41F 9/002; A61F 5/01; A61H 11/00

(57) ABSTRACT

An apparatus of self-adjustment belt with bumps is used during physical exercise, stretching, or just worn around a user's body to provide support, resistance, create compression, and offer a self-guided automatic adjustment. The apparatus provides constant conscious body awareness and can help enhance balance reactions and re-educate muscles. The bumps of the apparatus help relieve muscle tension by applying pressure and gentle massage to chiropractic trigger points of the user's body. The apparatus is equipped with sensors providing health data and monitoring activity or any health changes. Additionally, the apparatus provides heating and/or cooling to desired areas of the user's body. Further, the vibrators inside the bumps of the apparatus offer a better massage through vibration, which can also be light tapping, or electric stimulation, to relieve muscle tension and strain, tone muscles, and stimulate the circulatory system of the user.

The current application claims a priority to the U.S. Provisional Patent application Ser. No. 62/670,761 filed on May 12, 2018. The current application is filed on May 13, 2019 while May 12, 2019 was on a weekend.

### FIELD OF THE INVENTION

The present invention relates generally to garments and garment accessories. More specifically, the present invention relates to a self-adjustment belt with bumps that provides a user with spine support and massage, relives the user's muscle tension and strain, and stimulates the circulatory system of the user's body. Further, the present invention comprises a self-adjustment sleeve with bumps and a body suit with bumps.

### BACKGROUND OF THE INVENTION

Normally, chiropractic doctors treat more than just back pain, but many patients initially visiting a chiropractor look for relief from the pervasive condition of back pain. According to American Chiropractic Association (ACA), about 31 million Americans experience low back pain at any given time. Low back pain is the most common cause of job-related disability and a leading contributor to missed work days. About 80 percent of adults experience low back pain at some point in their lifetimes. Additionally, about 20 percent of people affected by acute low back pain develop chronic low back pain, a pain that persists for 12 weeks or longer, with persistent symptoms at one year, according to NIH' s the National Institute of Neurological Disorders and Stroke (NINDS) of the National Institute of Health (NIH). Further, the magnitude of the burden from low back pain has grown worse in recent years. In 1990, a study ranking the most burdensome conditions in the U.S. in terms of mortality or poor health as a result of disease put low back pain in sixth place; in 2010, low back pain jumped to third place. In the U.S., the peak age for back pain is between 25 and 45—in fact, it's the number one cause of disability for people under 45. In 2008, Americans spent $33.4 billion in 2008 treating the condition, not counting the estimated $66.6 billion lost in wages and decreased productivity. Many patients turn to back support belts and/or braces as an option to prevent or alleviate their pain.

A common type of belt or brace is neoprene belt that can be found in pharmacies or sporting goods store. Another common type of support is lumbar support bracing, which is often used in the management of low back pain, as well as in the workplace to prevent low back pain. Lumbar support bracing may be rigid or flexible, may be worn over or under a user's clothing, may or may not have shoulder straps, and may be worn all day, part of the day, or only during certain activities. Overall, the support belt and bracing provide benefits to the user when worn properly. The benefits include correcting deformity, facilitating healing, limiting movement, stabilizing the back, easing the pain, etc., which are especially helpful to the users who do heavy lifting on the job. Additionally, this type of belt can make a user transition back to work easier to manage after an injury. However, this common type of support belt may create more problems than the user initially has, especially if the support belt is not properly fitted or maintained. Specifically, the improperly fitted belts can cause skin irritation and lesions, lead to muscle atrophy seeing up the user's spine for injury with long-term use of the belts. Additionally, this type of belt may transfer the load through motion for one part of the back to other muscles resulting injury. Further, this type of belt may cause compression of the user's abdomen resulting in digestive gastro-intestinal disorders and increase the blood pressure and resting heart rate of the user. Some of the common support belts and braces do not have adjustable closures, thus, they can be restrictive and uncomfortable to wear if not adjusted well or position correctly on the user's body. Further, none of the existing support belts and braces provides massage therapy, one of the myofascial release therapy methods.

Myofascial release therapy deals with muscle pain, generally known as myofascial pain syndrome (MPS). MPS is one of the most common type of musculoskeletal disorders, which are the leading cause of disability among working-age adults, accounting for 34 percent of all workdays missed due to job-related injuries. There are several treatment options available for the patients suffering from MPS, including medications, trigger point injections, and ultrasonography. However, myofascial release therapy is so far the most effective non-invasive treatment for MPS.

MPS is a chronic condition characterized by inflammation and pain in the fascia which the layer of connective tissue that surrounds the musculoskeletal system. The muscles most often affected by myofascial pain are the ones used to maintain body posture including the neck, pelvic girdle, shoulders, and upper trapezius. Myofascial pain may also be accompanied by swelling of the joints. The resulting pain by MPS is caused by tender nodules of muscle fiber within the facial system called trigger points, or knots in the fascia. A large part of myofascial release includes targeting these trigger points and keeping steady pressure on them to help release them. Thus, mofascial release therapy is also called myofascial trigger point therapy, which encompasses a variety of massage techniques intended to stretch and loosen the fascia. Massage therapy, cupping, rolfing, structural integration, and instrument-assisted soft tissue mobilization (IASTM) are a few examples of myofascial release techniques, which are usually performed by an occupational therapist, registered massage therapist, chiropractor, osteopathic physician, or other health professionals who have undergone specialized training. When performed by a skilled professional, myofascial release therapy can have positive effects such as improving blood flow, encouraging lymphatic drainage, eliminating pain, and restoring motion to the musculoskeletal system. However, these therapy methods render the user time-consuming nature and financial constraint that hinder the wide application of the therapy methods.

There are many tools available that are designed to facilitate self-myofascial release by the user. The most popular myofascial release tools, also called trigger point tools for at-home massage include foam rollers, massage stick rollers, trigger point balls, and massage canes. These trigger point therapy tools help break up constricted fascia, reduce and restore flexibility to the muscles. However, there are many drawbacks and problems associated with all these popular myofascial release tools. First, these tools present a learning curve to the user that may be time-consuming and difficult. Second, these tools require hand effectiveness of the user in order to efficiently use them. Third, the use may have difficulty targeting certain areas of the user's body when performing self-myofascial release using any of these tools. Further, some of the tools may be restricted to certain areas of the user's body such as feet or neck, and some of the tools can cause discomfort.

Thus, it is an objective of the present invention to solve the aforementioned drawbacks, problems, and issues associated with support belts/braces and trigger point therapy tools. The present invention offers a versatile, self-adjustment belt with bumps that integrates trigger point therapy function into a self-adjustment support belt. The present invention provides the user with multiple benefits including adjustable and flexible compression, support of back and hips, muscle massage, heat and cold therapy, trigger point therapy, stretching aid, working out aid, self-adjustment and self-alignment, poor posture correction, increase of balance awareness, improved blood circulation, skin beautification, etc. This specific type of support is gentle not rigid, comfortable, flexible, intended to not only contour and massage pleasantly but to also send more sensory information to the brain simultaneously from the pressure points, encouraging self bracing, self-awareness, self correcting, and coordinating muscles working together.

### SUMMARY OF THE INVENTION

An apparatus of self-adjustment belt with bumps is used during physical exercise, stretching, or just worn around a user's body for a period of time to provide support, resistance, create compression, and offer a self-guided automatic adjustment. The apparatus provides constant conscious body awareness and can help enhance balance reactions and re-educate muscles.

The apparatus comprises a first layer, a core and a second layer. The second layer is an exterior layer and is made of a slightly flexible lightweight material, which helps a user develop good posture as it increases awareness of the user's body, and as a result it can help increase balance and mobility. The small degree of flexibility allows for comfort, movement, and breathing while the resulting compression will vary slightly during breathing. This layer of the adjustment belt is firm enough to offer support and compression like a second skin that supports the spine and body and helps reduce strain and stress during exercise and stretching.

The first layer is the interior layer of the apparatus and in direct contact with body of the user when in use. This first layer of the self-adjustment belt with bumps is made of bumps of a firmer material, firm enough to not collapse, in order to provide deep pressure and massage to the muscles, thus helping relieve stiffness and muscle tightness. This layer is firm, yet soft enough to be comfortable to the user; the firmness can vary, such as soft, medium, firm.

The core of the apparatus comprises a plurality of bumps that help relieve muscle tension by applying pressure and gentle massage to chiropractic trigger points of the user's body. The bumps help the user discover muscle tension, problem areas, restraint of motion, limited movement, tightness, soreness, pain, or other problems caused by body misalignment, and then the bumps help relax the muscle and regain proper alignment. The bumps of the apparatus are equipped with sensors that provide health data and activity monitoring and tracking. The sensors measure health data such as—pulse (heart rate), temperature, muscle tension (in order to detect the muscle knots and tense muscles), also such as step counter, caloric burn during the time it was worn or during a workout, etc. Additionally, these sensors extend inside the bumps to be able to provide data trends and monitor any health changes with connection to and communications with a control panel.

The apparatus provides heating and/or cooling elements in the core and/or bumps to desired areas of the user's body. The heating and/or cooling elements are made of a heat conductive material, a flexible electrical heating and cooling element inside the belt and extended inside the bumps. These heating/cooling elements cool or heat areas of the user's body. Further, the vibrators inside the bumps of the apparatus offer a better massage through vibration, which can also be light tapping, or electric stimulation, in order to relieve muscle tension and strain, tone muscles, and stimulate the circulatory system of the user.

The apparatus can also be made into a sleeve, or band, and/or even a full body suit incorporating circular bands and belts that are interconnected into a fashionable sporty body suit that can provide the same benefits to the full body of the user.

### BRIEF DESCRIPTION OF THE DRAWINGS

FIG. 1 is an isometric perspective view of the present invention.

FIG. 2 is a top view of the present invention.

FIG. 3 is a front view of the present invention indicating the direction of section cut A.

FIG. 4 is a cross sectional view of the present invention taken along section cut A in FIG. 3.

### DETAIL DESCRIPTIONS OF THE INVENTION

All illustrations of the drawings are for the purpose of describing selected versions of the present invention and are not intended to limit the scope of the present invention.

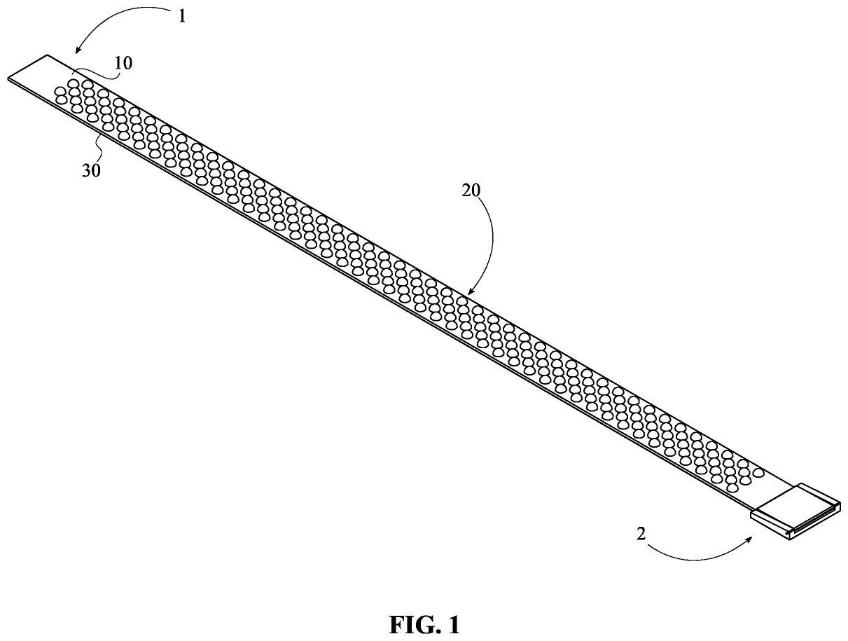

As can be seen in FIG. 1 to FIG. 4, in the preferred embodiment, the present invention comprises a self-adjustment belt with bumps for providing spine support and massage, relieving muscle tension and strain, and stimulating the circulatory system of human body. Specifically, the self-adjustment belt with bumps of the present invention comprises a belt body 1 and a belt closure 2. The belt closure 2 is terminally connected to the belt body 1 and allows a user to fasten the present invention to any part of the user's body. Additionally, the belt closure 2 provides an effective and efficient means to the user to adjust tension of the belt body 1 in order to achieve desired pressure on the specific muscle or group of muscles of the user and provide extra push to help adjust the vertebrae out of alignment. Further, the present invention provides constant pressure to the specific body area when worn by the user through the desired tension on the belt body 1 during movement of the user's body, and the slightly varying body pressure induced by inhaling and exhaling and movements of the user offers a self-guided automatic adjustment of the body. The belt closure 2 can be made of rigid and semi-rigid materials including, but not limited to, silicone, stainless steel, aluminum, metal, plastic, composite material, and/or any combination thereof. Additionally, the belt closure 2 includes, but is not limited to, buckle, clamp, clip buckle, ring buckle, grip buckle, wrestling buckle, latch buckle, pin buckle, side-release buckle, hook-and-loop fastener, zipper, Velcro®, or any other suitable closure type.

The belt body 1 of the present invention, as seen in FIG. 1, comprises a first layer 10, a core 20, and a second layer 30. Specifically, the core 20 is positioned between the first layer 10 and the second layer 30. The first layer 10 is normally in direct contact with the user's body when the user wears the present invention. Each of the first layer 10 and the core 20 is made of a firm and waterproof material including, but not limited to, silicone, rubber, gel, soft foam, plastic, composite, or any other suitable material. Further, each of the first layer 10 and the core 20 can comprise a heat conductive material. The second layer 30 is made of a lightly flexible lightweight material including, but not limited to, silicone, rubber, gel, woven fabric, leather, plastic, composite, or any other suitable material.

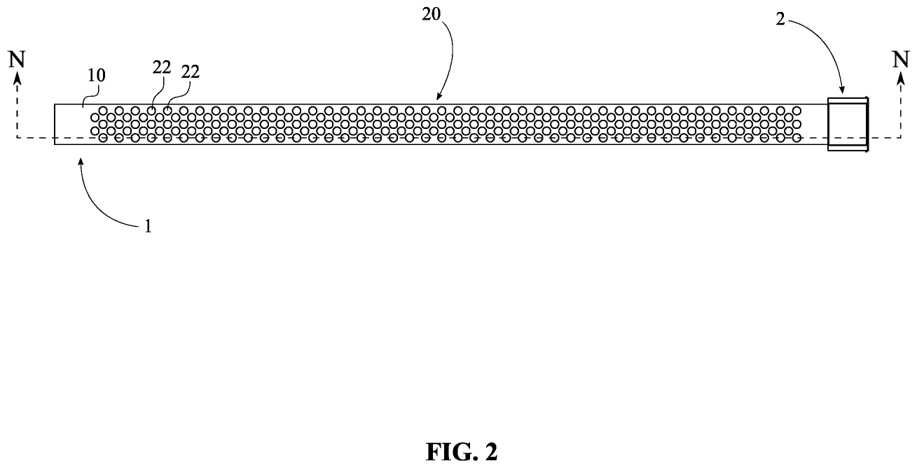

As can be seen in FIG. 2 to FIG. 4, the core 20 of the belt body 1 comprises a core layer 21, a plurality of bumps 22, a plurality of sensors 23, a plurality of vibrators 24, a plurality of heating/cooling elements 25, a plurality of tapping devices 26, and a plurality of electric stimulators 27. More specifically, the plurality of bumps 22 is distributed across the core 20. Each of the plurality of bumps 22 is positioned on the core layer 21, adjacent the first layer 10. Additionally, each of the plurality of bumps is projected out of the first layer 10. Each of the plurality of bumps 22 is made of a rigid material including, but not limited to, silicone, rubber, gel, soft foam, plastic, composite, or any other suitable material, to provide deep pressure and massage to the muscles of the user, thus, helping relieve stiffness, muscle tightness, and massage trigger points. The material used for each of the plurality of bumps 22 can be soft, medium firm, and firm so that the user can choose the most comfortable wear, to achieve the desired trigger point therapy effect. Additionally, the plurality of bumps 22 helps the user discover muscle tension, problem areas, restraint of motion, limited movement, tightness, soreness, pain, and/or other myofascial problems caused by body misalignment. The plurality of bumps 22 can help relax the muscle and regain the proper alignment. Further, each of the plurality of bumps 22 can comprise a shape including, but not limited to, spherical, semi-spherical, dome, triangular, oval, cone, diamond, rectangular prism, pyramid, or any other suitable shape. The density of distribution and total number of bumps of the plurality of bumps 22 can be varied by changing the distance or spacing between adjacent bumps to provide different pressure to different users and the same user under different situations. For example, closely spaced bumps of the plurality of bumps 22 can provide deeper pressure to a plurality of trigger points of the user's body than less closely spaced bumps.

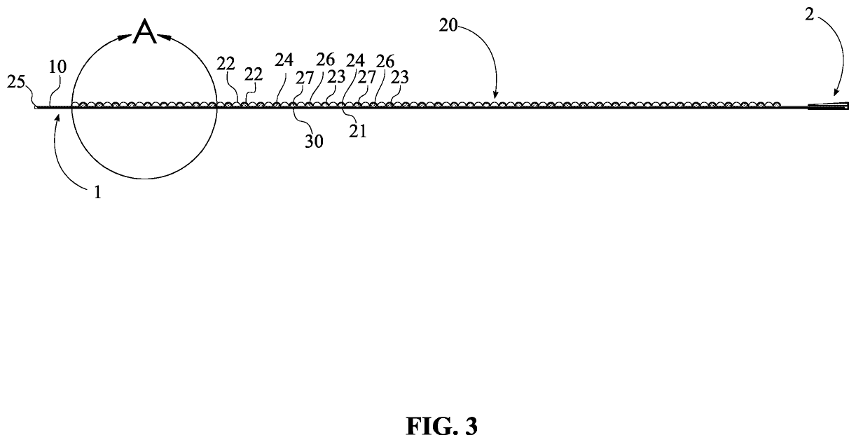

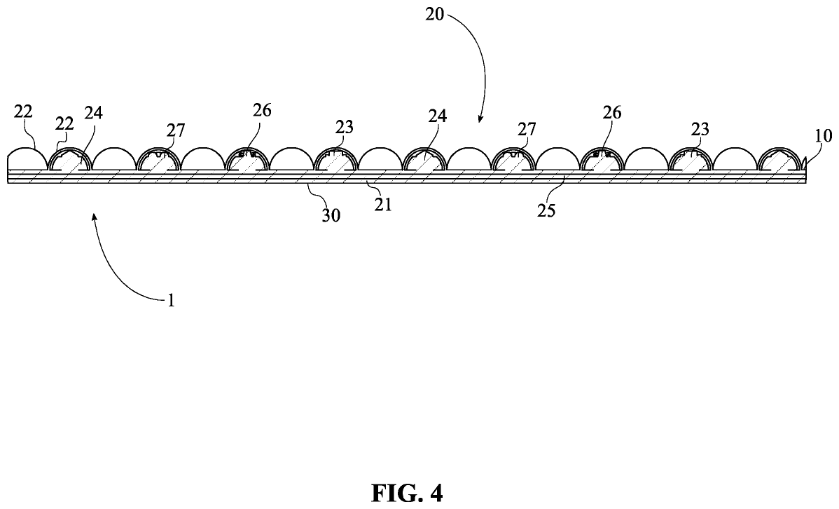

As can be seen in FIG. 3 to FIG. 4, the plurality of bumps 22 of the core 20 comprises the plurality of sensors 23. More specifically, each of the plurality of sensors 23 is positioned within one of the plurality of bumps 22. The plurality of sensors 23 can be fully covered by the plurality of bumps 22 or opened to the exterior depending on the type of sensor used on the belt body 1. The plurality of sensors 23 of the present invention can include, but is not limited to, a heart rate meter, a blood pressure meter, a temperature sensor, a muscle tension sensor, etc. Additionally, the plurality of sensors 23 can be wired to a power source and/or a control panel for the monitoring of a specific measurement including, but not limited to, heart rate, blood pressure, temperature, muscle tension, etc. Such communication connection can be wireless through a network, and the plurality of sensors 23 may be self-powered using batteries.

As can be seen in FIG. 3 to FIG. 4, the plurality of bumps 22 of the core 20 comprises the plurality of vibrators 24. More specifically, each of the plurality of vibrators 24 is positioned within one of the plurality of bumps 22. The plurality of vibrators 24 can include, but is not limited to, an electric vibrator, or any other suitable device that provides a better massage through vibration of the plurality of bumps 22. This can stimulate any part of the user's body to relieve muscle tension and strain, tone muscles, trigger points, and improve the circulatory system of the user. Additionally, the plurality of vibrators 24 can be wired to a power source and/or a control panel. Further, the plurality of vibrators 24 can be controlled through a wireless network and may be self-powered using batteries. In an alternative embodiment, the plurality of bumps 22 comprises the plurality of tapping devices 26, each of which is positioned within one of the plurality of bumps 22, as seen in FIG. 3 to FIG. 4. The plurality of tapping devices 26 functions the same as the plurality of vibrators 24 but has slightly different tapping effect that is more concentrated at the targeted muscle area for the user.

As can be seen in FIG. 3 to FIG. 4, the core 20 of the belt body 1 comprises the plurality of heating/cooling elements 25. More specifically, each of the plurality of heating/cooling elements 25 is positioned inside the core 20. Additionally, each of the plurality of heating/cooling elements 25 can be extended into one of the plurality of bumps 22. The plurality of heating/cooling elements 25 can include, but is not limited to, flexible heating/cooling band, heating/cooling strip, or any other suitable device that provide heating/cooling therapy to desired areas of the user's body for a better self-adjustment. The plurality of heating/cooling elements 25 can be powered using wire cables to an exterior power supply or batteries. Further, the plurality of heating/cooling elements 25 can be connected to a control panel through wire cables or a wireless network to offer convenient control of the heating/cooling of the belt body 1 to the user. This control includes, but is not limited to, on and off control, magnitude of heating/cooling to the desired body area, etc.

As can be seen in FIG. 3 to FIG. 4, the plurality of bumps 22 of the core 20 comprises the plurality of electric stimulators 27. More specifically, each of the plurality of electric stimulators 27 is positioned within one of the plurality of bumps 22. The plurality of electric stimulators 27 is a device used to provide the elicitation of muscle contraction using electric impulses and can be utilized as a strength training tool for athletes and in chiropractic massage therapy as a rehabilitation and preventive tool for partially or totally immobilized patients. The impulses generated by the device are delivered through electrodes on the skin near to the muscles being stimulated. The electrodes are generally pads that adhere to the skin. Further, the impulses mimic the action potential that comes from the central nervous system, causing the muscles to contract. Thus, the plurality of electric stimulators 27 can provide muscle warm up and contraction to specific trigger points and other body areas that the user desires to work on.

In some embodiments of the present invention, the belt body 1 of the self-adjustment belt with bumps is a sleeve, used on smaller circumference body parts, arms, legs, where the degree of flexibility is lower, which comprises a material including, but not limited to, silicone, gel, fabric, or any other suitable type of garment material. The present invention may or may not comprise the belt closure 2.

In other embodiments of the present invention, the belt body 1 of the self-adjustment belt with bumps is a body suit, which comprises of interconnected belts, sleeves, bands, interconnected inside a body suit made of a wearable including, but not limited to, fabric, cotton, silk, sports suit material, water-proof material, or any other suitable type of garment material. The present invention may or may not comprise the belt closure 2.

Although the invention has been explained in relation to its preferred embodiment, it is to be understood that many other possible modifications and variations can be made without departing from the spirit and scope of the invention as hereinafter claimed.

What is claimed is:

1. A self-adjustment belt with bumps for providing spine support and massage, relieving muscle tension and strain, and stimulating the circulatory system of human body, comprising:
a belt body;
the belt body comprising a first layer, a second layer and a core;
the core being positioned between the first layer and the second layer;
the core comprises a plurality of bumps;
the plurality of bumps being distributed across the core; and
each of the plurality of bumps being projected out of the first layer.

2. The self-adjustment belt with bumps for providing spine support and massage, relieving muscle tension and strain, and stimulating the circulatory system of human body as claimed in claim 1, comprising:
the plurality of bumps comprising a plurality of sensors;
each of the plurality of sensors being positioned within one of the plurality of bumps; and
the plurality of sensors being a heart rate meter.

3. The self-adjustment belt with bumps for providing spine support and massage, relieving muscle tension and strain, and stimulating the circulatory system of human body as claimed in claim 2, wherein the plurality of sensors being a blood pressure meter.

4. The self-adjustment belt with bumps for providing spine support and massage, relieving muscle tension and strain, and stimulating the circulatory system of human body as claimed in claim 2, wherein the plurality of sensors being a temperature sensor.

5. The self-adjustment belt with bumps for providing spine support and massage, relieving muscle tension and strain, and stimulating the circulatory system of human body as claimed in claim 2, wherein the plurality of sensors being a muscle tension sensor.

6. The self-adjustment belt with bumps for providing spine support and massage, relieving muscle tension and strain, and stimulating the circulatory system of human body as claimed in claim 1, comprising:
the plurality of bumps comprising a plurality of vibrators; and
each of the plurality of vibrators being positioned within one of the plurality of bumps.

7. The self-adjustment belt with bumps for providing spine support and massage, relieving muscle tension and strain, and stimulating the circulatory system of human body as claimed in claim 1, comprising:
the first layer comprising a heat conductive material;
core comprising a plurality of heating/cooling elements; and
each of the plurality of heating/cooling elements being positioned inside the core.

8. The self-adjustment belt with bumps for providing spine support and massage, relieving muscle tension and strain, and stimulating the circulatory system of human body as claimed in claim 7, wherein each of the plurality of heating/cooling elements being positioned inside one of the plurality of bumps.

9. The self-adjustment belt with bumps for providing spine support and massage, relieving muscle tension and strain, and stimulating the circulatory system of human body as claimed in claim 1, comprising:
the plurality of bumps comprising a plurality of tapping devices; and
each of the plurality of tapping devices being extended into one of the plurality of bumps.

10. The self-adjustment belt with bumps for providing spine support and massage, relieving muscle tension and strain, and stimulating the circulatory system of human body as claimed in claim 1, comprising:
the plurality of bumps comprising a plurality of electric stimulators; and
each of the plurality of electric stimulators being positioned within one of the plurality of bumps.

11. The self-adjustment belt with bumps for providing spine support and massage, relieving muscle tension and strain, and stimulating the circulatory system of human body as claimed in claim 1, comprising:
a belt closure; and
the belt closure being terminally connected to the belt body.

12. The self-adjustment belt with bumps for providing spine support and massage, relieving muscle tension and strain, and stimulating the circulatory system of human body as claimed in claim 1, wherein belt body is a sleeve.

13. The self-adjustment belt with bumps for providing spine support and massage, relieving muscle tension and strain, and stimulating the circulatory system of human body as claimed in claim 1, wherein belt body is a body suit.
